(19) United States
(12) Patent Application Publication
(10) Pub. No.: US 2005/0169874 A1
(43) Pub. Date: Aug. 4, 2005

(54) Polyurethane emulsifiers

(76) Inventors: Albert A. Zofchak, Holmdel, NJ (US); John Carson, Union City, NJ (US)

Correspondence Address: Henry D. Coleman, 714 Colorado Avenue, Bridgeport, CT, 06605-1601, US

(21) Appl. No.: 11/035,634

(22) Filed: Jan. 14, 2005

Related U.S. Application Data
(60) Provisional application No. 60/541,317, filed on Feb. 3, 2004.

(51) Int. Cl. A61K 7/06; A61K 7/11

(52) U.S. Cl. 424/70.17; 528/74.5

(57) ABSTRACT

The invention provides polymeric emulsifiers that: are useful in the manufacture of personal care products such as skin creams, sunscreens, hair conditioners and shampoos; have similar aesthetic properties as conventional surfactants when used in personal care product applications; and that are relatively free of the drawbacks associated with known anionic or nonionic polymeric emulsifiers. In certain embodiments, the invention provides o/w or w/o emulsions comprising cationic polyurethane emulsifiers (surfactants) or combinations of cationic polyurethane emulsifiers and conventional low HLB emulsifiers. Emulsions of the invention are useful as components of personal care products such as hair and skin care products and are functional and stable over a wide pH range.

### RELATED APPLICATIONS

This application claims the benefit of priority of provisional application U.S. 60/541,317, filed Feb. 3, 2004, the contents of said application being incorporated by reference herein in its entirety.

### FIELD OF THE INVENTION

The invention provides polymeric emulsifiers that: are useful in the manufacture of personal care products such as skin creams, sunscreens, and shampoos; have similar aesthetic properties as conventional surfactants when used in personal care product applications; and that are relatively free of the drawbacks associated with known anionic, nonionic or cationic polymeric emulsifiers. In one embodiment, the invention provides o/w or w/o emulsions comprising cationic polyurethane emulsifiers (surfactants) or combinations of cationic polyurethane emulsifiers and conventional low HLB emulsifiers. Emulsions of the invention are useful as components of personal care products such as hair and skin care products and are functional and stable over a wide pH range.

### BACKGROUND OF THE INVENTION

Emulsions are used in personal care products such as sun screens, skin creams, and shampoos in order to combine the moisturizing and skin softening effects of water, water soluble ingredients and humectants with the soothing and protecting properties of oils. Typically, emulsions used in personal care products are formed by dispersing either discrete droplets of oil within a water phase (oil-in-water emulsions or “o/w emulsions”) or discrete droplets of water within an oil phase (water-in-oil emulsions or “w/o emulsions”). Surfactants are used in making emulsions and serve to reduce the amount of energy needed to disperse one of the phases within the other by making the surface of the dispersed phase more compatible with the continuous or external phase. Surfactants with lower water solubility (or higher oil solubility) tend to be w/o emulsifiers, while those with higher water solubility (lower oil solubility) tend to produce o/w emulsions.

Water versus oil solubility of a surfactant is characterized by its Hydrophilic/Lipophilic Balance (“HLB”). More water soluble surfactants (o/w directing) have high HLB's ranging from about 13 to 20 or more. Less water soluble surfactants (w/o directing) have low HLB's in approximately the 0 to 6 range. A stable and aesthetically pleasing o/w emulsion useful in personal care products can be made by using a combination of o/w and w/o emulsifiers (or high HLB and low HLB surfactants). The water soluble surfactant (high HLB) reduces surface tension and, with the w/o (low HLB) emulsifier and water, forms a hydrated, lamellar material in the water phase that increases viscosity and creates a barrier around the dispersed oil droplets. This barrier prevents coalescence of the oil droplets and stabilizes the emulsion.

Polymeric emulsifiers (surfactants) such as the Pemulen®products (INCI name: C10-30 Alkyl Acrylates Crosspolymer) are used in personal care products because they: are extremely mild and non-irritating to skin and mucous membranes; do not penetrate into the skin to cause irritation; do not require heat to be melted; and once applied to a surface and dehydrated, they become poorly functional as surfactants and have little ability to re-emulsify the oil phase from the surface. Natrosol® Plus CS (INCI name Cetyl Hydroxyethyl Cellulose) is a nonionic polymeric emulsifier that does not require neutralization and that is used in personal care products. Such polymeric emulsifiers suffer from the following disadvantages when compared to conventional surfactants.

The polymeric emulsifiers do not have the same aesthetic properties as those made using conventional surfactants (e.g., do not feel as smooth and lubricious). An emulsion made with the polymeric emulsifiers is shear thinning and therefore flows easily on the skin as it is rubbed. As the emulsion loses water during rubbing, a point is reached (“the break point”) where the emulsion structure loses integrity. In a soap emulsion at the break, the emulsion's oily components and surfactants are rapidly deposited on the skin. This results in a pronounced increase in rubbing friction that leads to the desirable sensation that the skin lotion or cream has been “rubbed in.” In a typical polymeric emulsion, at the break point, the emulsifier loses functionality leaving in the oil phase materials to be rubbed about on the skin. This does not result in a high friction “rubbed in” impression, but rather that one has simply applied oil to their skin.

Cationic emulsifiers used in personal care products have a distinct break point, but leave a dry sensation upon application (rub in) that can cause skin to feel powdered. Rewetting of a dried cationic emulsion with water will often produce “beading” of the water droplets as the emulsifier resists wetting. Further, known nonionic and cationic emulsifiers can create a gel-like, slippery sensation upon application, that, as skin dries, becomes more oily in feel.

Thus, the need exists for polymeric emulsifiers that: are useful in the manufacture of personal care products such as skin creams, sunscreens, and hair conditioners; have similar aesthetic properties as conventional surfactants when used in personal care product applications; and that are relatively free of the drawbacks associated with the known anionic and nonionic polymeric emulsifiers.

### SUMMARY OF THE INVENTION

The invention provides polymeric emulsifiers that: are useful in the manufacture of personal care products such as skin creams, sunscreens, and shampoos; have similar aesthetic properties as conventional surfactants when used in personal care product applications; and that are relatively free of the drawbacks associated with known anionic or nonionic polymeric emulsifiers. In one embodiment, the invention provides o/w emulsions comprising cationic polyurethane emulsifiers (surfactants) or combinations of cationic polyurethane emulsifiers and conventional low HLB emulsifiers. Emulsions of the invention are useful as components of personal care products such as hair and skin care products and are functional and stable over a wide pH range.

Cationic polyurethane emulsifiers of the invention are formed by:
(a) reacting approximately equimolar amounts of triethanolamine with a fatty acid to form a dihydroxyethyl aminoethyloxy fatty acid ester;
(b) reacting approximately equimolar amounts of the dihydroxyethyl aminoethyloxy fatty acid ester with a diisocyanate to form a urethane polymer; and
(c) reacting the urethane polymer with quaternizing agent.

In another embodiment, the invention provides o/w emulsions comprising:
(1) a polyurethane emulsifier formed by:
(a) reacting approximately equimolar amounts of triethanolamine with a fatty acid to form a dihydroxyethyl aminoethyloxy fatty acid ester;
(b) reacting approximately equimolar amounts of the dihydroxyethyl aminoethyloxy fatty acid ester with a diisocyanate to form a urethane polymer; and
(c) reacting the urethane polymer with quaternizing agent;
(2) a non-aqueous phase comprising one or more of an emollient, nonionic surfactant, cationic surfactant or non-polyurethane emulsifier; and
(3) water,
   wherein either the non-aqueous phase is dispersed in water or the water is dispersed in the non-aqueous phase to form an oil-in-water emulsion.

In preferred embodiments, the fatty acid is selected from the group consisting of behenic, erucic, arachidic, gadoleic, stearic, oleic, hydroxystearic, palmitic, and palmitoleic acids, the diisocyanate is isophorone diisocyanate, and the quaternizing agent is selected from the group consisting of alkyl halides, dimethyl sulfate, and diethyl sulfate.

Cationic polyurethane emulsifiers and o/w emulsions of the invention are useful in the formulation of personal care products such as skin and hair conditioners, skin creams, sunscreens, and shampoos.

Compositions according to the present invention bring novel unanticipated properties which can be traced to the presence of the “urethane linkage” within the emulsifying polymer. This linkage synergistically increases adhesion to hair and skin contacting formulations, while maintaining other favorable attributes. In addition, the present compositions which are based on polyurethane chains allow the manufacturer a considerable degree of flexibility for formulation due to the ability to vary viscosity and molecular weight characteristics of the composition—a function of the polymeric chain size and substituents.

These and other features of the invention are described further in the following detailed description.

### DETAILED DESCRIPTION OF THE INVENTION

As used herein, the following terms have the following respective meanings. Other terms that are used to describe the present invention have the same definitions as those generally used by those skilled in the art. Specific examples recited in any definition are not intended to be limiting in any way.

“Alkyl” refers to a fully saturated monovalent hydrocarbon radical containing carbon and hydrogen which may be a straight chain, branched, or cyclic. Generally, although not necessarily, alkyl groups herein contain 2 to about 24 carbon atoms. Examples of alkyl groups are methyl, ethyl, n-butyl, n-heptyl, isopropyl, 2-methylpropyl, cyclopropyl, cyclopropylmethyl, cyclobutyl, cyclopentyl, cyclopentylethyl and cyclohexyl.

“Alkenyl” refers to a branched or unbranched hydrocarbon group typically although not necessarily containing 2 to about 24 carbon atoms and at least one double bond, such as ethenyl, n-propenyl, isopropenyl, n-butenyl, isobutenyl, octenyl, decenyl, and the like.

The term “effective amount” is used throughout the specification to describe concentrations or amounts of compounds according to the present invention which effect an intended result such as being effective to synthesize a compound or composition according to the present invention or in conveying a desired trait or in effecting a desired condition such as emulsification, clarification, adhesion, melting point modification or solubility to a formulation of a cosmetic, toiletry or personal care product.

The term “approximately equimolar” is used within context of producing cationic polyurethane emulsifiers according to the present invention to denote amounts of reactants which are essentially equivalent, but may vary somewhat within the context of the reaction to enhance yield and purity of the desired compound or composition. Preferably, approximately equimolar refers to a molar ratio of each of the reacants which at least 80%, at least about 90%, at least about 95%, at least about 98%, at least about 99% and the same as (100%) of the molar ratio of the other reactants within a chemical reaction.

“Emollients” are dermatologically acceptable compositions that tend to lubricate the skin, increase the smoothness and suppleness of the skin, prevent or relieve dryness of the skin, and/or protect the skin. Emollients are typically water-immiscible, oily or waxy materials. A wide variety of suitable emollients are known and may be used herein. These include emollients may be selected from one or more of the following classes: triglyceride esters which include, but are not limited to, vegetable and animal fats and oils such as: castor oil, cocoa butter, safflower oil, cottonseed oil, corn oil, olive oil, cod liver oil, almond oil, avocado oil, palm oil, sesame oil, squalene, kikui oil, soybean oil and tricapryl, tricaprylyl and triisostearyl esters; mineral oils, petrolatums, silicone oils composed of dimethylpolysiloxanes; Acetoglyceride esters, such as acetylated monoglycerides; Ethoxylated glycerides, such as ethoxylated glyceryl monostearate; Alkyl esters of fatty acids having 10 to 24 carbon atoms which include, but are not limited to, methyl, isopropyl, butyl, hexyl, isohexyl, octyl, isooctyl, decyl, isodecyl and hexadecyl esters of fatty acids such as hexyl laurate, isohexyl laurate, isohexyl palmitate, isopropyl palmitate, isopropyl myristate, methyl palmitate, decyloleate, isodecyl oleate, hexadecyl stearate, decyl stearate, isopropyl isostearate and methyl isostearate; diesters formed from the above listed alcohols and diacids such as: adipic and sebacic; monoesters formed from fatty alcohols and mono functional acids such as: lauryl lactate, myristyl lactate, cetyl acetate and cetyl lactate; Alkenyl esters of fatty acids having 10 to 24 carbon atoms such as oleyl myristate, oleyl stearate, and oleyl oleate; Fatty acids having 10 to 24 carbon atoms such as pelargonic, lauric, myristic, palmitic, stearic, isostearic, hydroxystearic, oleic, linoleic, ricinoleic, arachidic, behenic, and erucic acids; Fatty alcohols having 10 to 24 carbon atoms such as lauryl, myristyl, cetyl, hexadecyl, stearyl, isostearyl, hydroxystearyl, oleyl, ricinoleyl, behenyl, erucyl, and 2-octyl dodecanyl alcohols; Lanolin and lanolin derivatives such as lanolin, lanolin oil, lanolin wax, lanolin alcohols, lanolin fatty acids, isopropyl lanolate, ethoxylated cholesterol, propoxylated lanolin alcohols, acetylated lanolin alcohols, lanolin alcohols linoleate, lanolin alcohols ricinoleate, acetate of lanolin alcohols ricinoleate, acetate of ethoxylated alcohols-esters, hydrogenolysis of lanolin, ethoxylated hydrogenated lanolin, and liquid and semisolid lanolin absorption bases; Polyhydric alcohol esters such as ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol polyfatty esters, ethoxylated glyceryl monostearate, 1,2-butylene glycol monostearate, 1,2-butylene glycol distearate, pentaerythritol tetra caprate, pentaerythritol tetra caprylate, pentaerythritol tetra isostearate, sorbitan fatty acid esters, and polyoxyethylene sorbitan fatty acid esters; Wax esters such as beeswax, spermaceti, myristyl myristate, stearyl stearate and jojoba oil; Beeswax derivatives such as polyoxyethylene sorbitol beeswax which are reaction products of beeswax with ethoxylated sorbitol of varying ethylene oxide content, forming a mixture of ether esters; Vegetable waxes including, but not limited to, carnauba and candelilla waxes; Phospholipids such as lecithin and derivatives; Sterols including, but not limited to, cholesterol and cholesterol fatty acid esters; and Amides such as fatty acid amides, ethoxylated fatty acid amides, and solid fatty acid alkanolamides.

The term “fatty acid” is used herein to describe a C₂ to C₂₅ carboxylic acid, preferably a C₁₀ to C₂₂ fatty acid. The alkyl portion may be saturated or unsaturated, branched or unbranched.

The term “personal care product” is used throughout the specification to describe a cosmetic or toiletry product which produces its effect through a physicochemical interaction of the product with the user (in contrast to a mechanical product) which is preferably used on or in contact with the hair, skin and/or nails and which include effective concentrations of one or more of the compositions according to the present invention. Personal care products include, for example, cosmetics, floating bath oils, after shaves, creams, lotions, deodorants, including stick deodorants, pre-electric shave lotions, after-shave lotions, antiperspirants, shampoos, conditioners and rinses and related products, among others, including skin care products, eye makeups, body shampoos, protective skin formulations, lipsticks, lip glosses, after-bath splashes, presun and sun products, including sunscreens and include chemical components, such as emollients, preservatives, opacifying agents, surfactants, dyes, hair conditioning agents, thickeners, gelling agents, stiffening agents, anti-perspirant agents, deodorizing agents, foaming agents, fragrances, solubilizers, solvents, sunscreen agents, among numerous others depending upon the personal care product, in combination with the present invention in amounts effective to produce the personal care product. Virtually any chemical product which comes into contact with the hair or skin and which may include effective amounts or concentrations of one or more of the compositions according to the present invention may be considered a personal care product according to the present invention.

The term “diisocyanate” refers to a diisocyanate compound used in the present invention to react with free hydroxyl groups on the triethanolamine fatty acid ester to form (poly)urethane compounds which may be quaternized to produce the cationic polyurethane emulsifiers. The term “diisocyanate” is used throughout the specification to describe a linear, cyclic or branch-chained hydrocarbon having two free isocyanate groups. C₄ to C₂₄ diisocyanate compounds are contemplated for use in the present invention, with preferred diisocyanates being C₆ to C₁₂ diisocyanates. The term “diisocyanate” also includes halogen substituted linear, cyclic or branch-chained hydrocarbons having two free isocyanate groups. Exemplary diisocyanates include, for example, isophorone diisocyanate, m-phenylene-diisocyanate, p-phenylenediisocyanate, 4,4-butyl-m-phenylene-diisocyanate, 4-methoxy-m-phenylenediisocyanate, 4-phenoxy-m-phenylenediisocyanate, 4-chloro-m-phenyldiisocyanate, toluenediisocyanate, m-xyenediisocyanate, p-xylenediisocyanate, 1,4-napthalenediisocyanate, cumene-1,4-diisocyanate, durene-diisocyanate, 1,5-napthylenediisocyanate, 1,8-napthylenediisocyanate, 1,5-tetrahydronapthylenediisocyanate, 2,6-napthylenediisocyanate, 1,5-tetrahydronapthylenediisocyanate; p,p-diphylenediisocyanate; 2,4-diphenylhexane-1,6-diisocyanate; methylenediisocyanate; ethylenediisocyanate; trimethylenediisocyanate, tetramethylenediisocyanate, pentamethylenediisocyanate, hexamethylenediisocyanate, nonamethylenediisocyanate, decamethylene-diisocyanate, 3-chloro-trimethylenediisocyanate and 2,3-dimethyltetramethylenediisocyanate, among numerous others. Isophorone diisocyanate is used the preferred diisocyanate used in the present invention.

Emulsions are two-phase systems in which one of the phases is finely and uniformly dispersed within the other. Mixing the two phases with an appropriate surfactant emulsifier, which also functions to stabilize the emulsion, initially makes the dispersion. For example, sun screening ingredients are usually solubilized in an oil phase, that is then added to a surfactant containing water phase to make an emulsion.

The term “quaternizing agent” is used throughout the specification to describe compounds which are used to react with tertiary amines to produce quarternary salts according to the present invention. Quaternary salts are salts which are produced when a tertiary amine is reacted with a quaternizing agent to produce a quaternary amine (quaternium) which is substituted with four carbon-containing groups. The quaternary amine produced is cationic and is generally found complexed with an anionic group or “counterion”, which is generally, but not always, derived from the quaternizing agent used to produce the quaternary amine. Exemplary quaternizing agents for use in the present invention include: dimethyl sulfate, diethyl sulfate, methyl bromide, benzyl chloride, ethyl benzyl chloride, methyl benzyl chloride, dichloroethyl ether, epichlorohydrin, ethylene chlorohydrin, methyl chloride, monochloroacetic acid and allyl chloride, among others, such that the group reactive with the amine produces a

$$Q - {N}^{+} - {R}_{3}$$

(the three R groups being those groups which are bonded to the amine prior to quaternization, the fourth group Q being the quaternizing group) group with the amine and the positively charged quaternary amine group is complexed with an anionic group or counterion, which is represented as R⁵⁻. The quaternizing group is that group which results from quaternizing the tertiary amine with the quaternizing agent and includes, for example, methyl, ethyl, propyl, benzyl, phenyl, alkyl benzyl, allyl and numerous other groups. The counterion may be any group which is anionic and is compatible with the chemistry of the present invention and preferably is an anionic chloride, bromide, iodide, fluoride, carboxylate (from, for example the use of chloroacetic acid or sodium monochloroacetate as the quaternizing agent to provide an acetate which can provide both a quaternium group as well as the counterion) sulfate (mono- or di-anion, preferably alkyl substituted mono-anion such as methyl or ethyl sulfate, more preferably ethyl sulfate) and phosphate (mono-, di- and tri-anion, preferably tri-anion), among numerous others, with anionic chloride and sulfate (alkyl substituted mono-anion) being the preferred counterion R⁵.

Cationic polyurethane emulsifiers and o/w and w/o emulsions of the invention can be combined in personal care products in accordance with the invention with a variety of supplementary surfactant systems. Such surfactants include those that function as detergents to clean the hair. Conventional surfactants such as anionic, cationic and amphoteric surfactants can be used. In shampoo formulations, sodium-based surfactants are preferred over ammonium-based surfactants. Commercial sources of such surfactants can be found in McCutcheon's EMULSIFIERS AND DETERGENTS, North American Edition, 1984, McCutcheon Division, MC Publishing Company, the complete disclosure of which is hereby incorporated by reference. The amount of surfactant can range from about 1% to about 70% on a weight percentage basis, more typically from about 2% to about 50%. Preferred surfactants include ammonium lauryl sulfate, ammonium laureth sulfate, triethylammonium lauryl sulfate, triethylammonium laureth sulfate, triethanolammonium lauryl sulfate, triethanolammonium laureth sulfate, monoethanolammonium lauryl sulfate, monoethanolammonium laureth sulfate, diethanolammonium lauryl sulfate, diethanolammonium laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolammonium lauryl sulfate, triethanolammonium lauryl sulfate, monoethanolammonium cocoyl sulfate, monoethanolammonium lauryl sulfate, sodium tridecyl benzene sulfonate, sodium dodecyl benzene sulfonate, and cocoamphocarboxyglycinate. Ammonium laureth sulfate and sodium laureth sulfate are particularly preferred.

Cationic polyurethane emulsifiers and o/w and w/o emulsions of the invention can be combined in personal care products in accordance with the invention with a variety of auxiliary conditioning agents such as quaternary ammonium compounds, amines, amine salts and other cationic polymers. Among the quaternary ammonium compounds suitable for use are quaternary ammonium hydroxides, such as methyl triethanol ammonium hydroxide and tetraethyl ammonium hydroxide. Preferred auxiliary conditioning agents include polyquaternium 10 or behentrimonium methosulfate. Such auxiliary conditioning agents can be used in an amount of from about 0.1% to about 3.0% on a weight percentage basis, preferably from about 0.5% to about 2.0%.

Cationic polyurethane emulsifiers and o/w and w/o emulsions of the invention can be combined in personal care products in accordance with the invention with a variety of emollients including, but are not limited to, mineral oil and petrolatum. Other emollients may include cetyl or stearyl alcohol, paraffin or lanolin alcohol. Emollients are generally employed in the formulations of the instant invention in a weight percentage range of from about 5% to about 45%, preferably from about 7.5% to about 20%. Examples of suitable humectants include, but are not limited to, propylene glycol, hexylene glycol, glycerin and sorbitol. As a general guide, humectants are used in a weight percentage range of from about 1% to about 20%, preferably from about 2% to about 10%.

Cationic polyurethane emulsifiers and o/w and w/o emulsions of the invention can be combined in personal care products in accordance with the invention with a variety of supplementary emulsifying agents to facilitate dispersion and suspension of the components, and render a creamy and lubricous consistency to the composition. Non-limiting examples of emulsifying agents suitable for use include alkoxylated alcohols and fatty alcohols, such as stearyl, cetyl and cetearyl alcohols, ethoxylated sorbitan esters, ethoxylated lanolin and derivatives thereof. As a general guide, supplementary emulsifiers can be used in amounts of about 1% to about 16% on a weight percentage basis, preferably from about 2% to about 12%, and more preferably from about 4% to about 10%.

In the instant invention, cationic polyurethane emulsifiers and o/w and w/o emulsions of the invention are included in personal care products/formulations in effective amounts, i.e., amounts which produce an intended effect. The amount of cationically charged polyurethane emulsifiers and o/w and w/o emulsions of the invention generally ranges from about 0.5% to about 10% by weight or more of personal care formulations according to the present invention. In preferred embodiments, cationically charged polyurethane emulsifiers and o/w and w/o emulsions of the invention are included in amounts ranging from about 0.5% to about 5% by weight of the final personal care formulations according to the present invention. In preferred sunscreen formulations, cationically charged polyurethane emulsifiers and o/w and w/o emulsions of the invention are included in amounts ranging from about 5% to about 10% by weight of the final formulation.

Cationic polyurethane emulsifiers and o/w and w/o emulsions of the invention can be combined in personal care products in accordance with the invention with a variety of supplementary thickeners such as natural gums such as tragacanth, xanthan, acacia and locus bean, and synthetic gums such as hydroxypropylmethylcellulose and hydroxyethyl cellulose. Polyvinyl alcohols can also be used. Alkanolamides, “super” amides and the glycol or glycerol stearates may also be used.

Cationic polyurethane emulsifiers and o/w and w/o emulsions of the invention can be combined in personal care products in accordance with the invention with other optional additives including preservatives, sequestrates, antioxidants such as sodium sulfite, chelating agents such as EDTA, suspending agents, fragrances or perfumes, herbal extracts, sunscreens, and pH control agents such as citric acid. These additives are usually present in an amount of less than 5% on a weight percentage basis. In a shampoo, an antidandruff component, e.g., selenium sulfide, may also be included at an effective level.

Properties and characteristics of the cationic polyurethane emulsifiers and o/w and w/o emulsions of the invention used in the present invention that make them especially useful as components of personal care, cosmetic, and toiletry application products include the following: extremely low order of toxicity and irritation; low color; excellent compatibility in cosmetic formulations; solubility with amides, sulfonates sulfosuccinates, and sulfobetaines; nonrancidification; coupling characteristics; and solubility/dispersibility in water, glycols and lower molecular weight alcohols.

Cationic polyurethane emulsifiers and o/w and w/o emulsions of the invention contribute to one or more of the following desirable attributes of a personal care product:
1) Increased sheen in hair conditioners.
2) Increased adhesion to the hair shaft wherein the urethane linkage exhibits synergistic effect with the quaternized tertiary amine functionality.
3) The increased adhesion characteristics directly attributable to the “urethane linkages” within the polymers of the present invention extends the effectiveness of a given skin and hair contacting formulation.
4) Better wet and dry combing characteristics are attainable by the introduction of the present compositions.
5) The “urethane linkages” of the present invention and the adhesion they promote to the hair shaft prolongs the effectiveness of the antistatic over and above quaternaries which have been in use.
6) The introduction of the present invention to skin and hair contacting formulations bring a velvety softness and longer lasting conditioning which is superior to that which is attainable with prior art compositions.
7) Increased color compatibility and conditioning for hair color products.

By way of example, the esterification of triethanolamine with the fatty acid moiety can be carried out in acid catalyzed reactions using well-known catalysts such as methane sulfonic acid, p-toluene sulfonic acid and hypophosphorous acid, among numerous other acids, at temperatures ranging from about 100° C. to about 250° C., from preferably about 115° C. to 225° C., generally at ambient pressure. Transesterification of a triglyceride such as castor oil with a triethanolamine can be conducted at temperatures ranging from 85° C. to 150° C. with a caustic (base) catalyst such as sodium hydroxide, sodium methylate, etc. at ambient pressures. The ratio of fatty acid to the triethanolamine may be varied from 1 mole of triethanolamine to 3 moles of the fatty acid moiety; preferably, 1 mole of triethanolamine is reacted with 1 mole of fatty acid. By varying the weight ratio of triethanolamine to fatty acid, one or ordinary skill can produce a mono-, di- or triesterified triethanolamine.

The urethane reaction of the present invention may occur at the unreacted hydroxyl groups of the triethanolamine or may take place at the hydroxyl groups of the fatty acid of the triethanolamine fatty acid ester. The urethane reactions will occur at a temperature range of approximately 60° C. to approximately 140° C. or higher. The ratio of the diisocyanate to triethanolamine ester may range from about 1:3 to about 3:1 depending upon the number of hydroxyl groups which occur in the triethanolamine and fatty acid substitutuents of the ester as well as the desirability of obtaining compositions which are more chain-extended in character rather than crosslinked. A molar ratio of diisocyanate to triethanolamine ester ranging from about 1:1 to about 1:2 is preferred, with a ratio of about 1:1 to about 1:1.2 in certain more preferable aspects of the present invention.

The resulting polymeric urethane ester is usually a fairly viscous liquid or a solid product with activity of 100%, although the viscosity may be adjusted accordingly by changing the molar ratio of the reactants, the type of fatty acid used, and the type of diisocyanate used as well as the molar ratios of each of those components used. The quaternarization is carried out in a procedure in which the present invention is heated to a temperature of about room temperature to about 100° C. or more, preferably, about 60° C. to about 85° C. preferably in the absence of a diluent or solvent and the quarternarizing agent such as diethyl sulfate, dimethyl sulfate, benzyl chloride, among others as set forth in greater detail herein, is slowly added. Once the quaternization is completed, an inert diluent such as propylene glycol, hexylene glycol or other pharmaceutically or cosmetically acceptable diluent may be added to reduce viscosity of the higher molecular derivative. Alternatively, and depending upon the molecular weight, crosslink density and viscosity of the polyurethane ester, quaterniziaton may occur in the presence of a diluent or solvent in addition to the quaternizing agent in order to provide for efficient reaction conditions. One of ordinary skill will know how to readily adjust the conditions by way of temperature and use of solvent, in order to provide compositions according to the present invention.

Final quaternized compositions according to the present invention have been found to be compatible with the esters (emollients), surfactants, emulsifiers and diluents that are used in skin and hair contacting formulations that find use in the cosmetic, toiletry and personal care industries. In addition, the compositions have a low irritation index and are compatable with the skin.

For example, a cationic polyurethane emulsifier of the invention can be made as follows. In accordance with the following reaction scheme, approximately equimolar amounts of triethanolamine are reacted with a fatty acid to form a dihydroxyethyl aminoethyloxy fatty acid ester (1). The dihydroxyethyl aminoethyloxy fatty acid ester (1) is reacted with an approximately equimolar amount of isophorone diisocyanate to form a urethane polymer (2). The urethane polymer (2) is reacted with approximately one mole of diethyl sulfate per equivalent of tertiary amine to form a quaternized product (3): 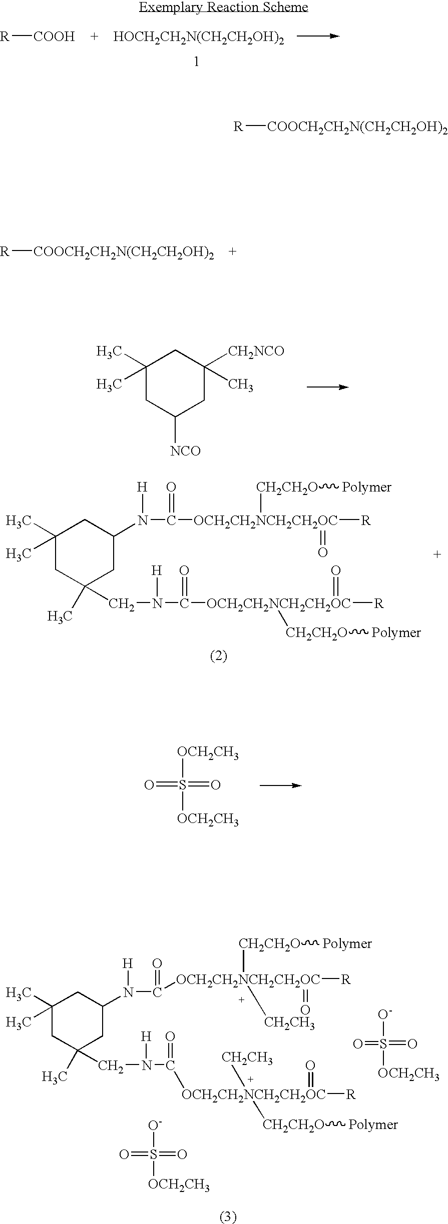
where R is a C₁₅-C₂₄ alkyl or alkenyl group.

The following provides some examples of formulations comprising cationic polyurethane emulsifiers of the invention.

Cationic polyurethane emulsifiers of the invention can be used as a co-emulsifier in an emulsifying wax mixture containing from about 10% to about 75% of the total formulation and from about 90% to about 25% of w/o (low HLB) emulsifiers chosen from the group of surfactants having an HLB (hydrophile/lipophile balance) of less than about 6. Such surfactants include but are not limited to: fatty alcohols, low mole ethoxylates of fatty alcohols, fatty acids, low mole ethoxylates of fatty acids, mono and di glyceryl esters, mono glycol esters, ethoxylated mono and di glyceryl esters, ethoxylated mono glycol esters and fatty amides that optionally contain an auxilliary high HLB surfactant.

Cationic polyurethane emulsifiers of the invention can be used as a co-emulsifier in an emulsion of oily material in water containing from 0.5% to 5% of the cationically charged polyurethane emulsifier and 1% to 10% of w/o (low HLB) emulsifiers chosen from the group of surfactants having an HLB (hydrophile/lipophile balance) of less than 6. These include but are not limited to: fatty alcohols, low mole ethoxylates of fatty alcohols, fatty acids, low mole ethoxylates of fatty acids, mono and di glyceryl esters, mono glycol esters, ethoxylated mono and di glyceryl esters, ethoxylated mono glycol esters and fatty amides.

Cationic polyurethane emulsifiers of the invention can be used in formulations useful as hair conditioners.

Cationic polyurethane emulsifiers of the invention can be used in formulations that are thickened through the use of other materials such as cationic surfactants cationic polymers other than those of the invention, nonionic surfactants, nonionic polymers, amphoteric surfactants, amphoteric polymers useful as emulsifiers, and amphoteric polymers that are useful as a hair conditioner.

Cationic polyurethane emulsifiers of the invention can be used as an emulsifier for ultraviolet light absorbing materials used in sunscreens.

Cationic polyurethane emulsifiers of the invention can be used with nonionic, other cationic, or amphoteric polymers to control viscosity.

These and other aspects of the invention are described further in the following examples, which are illustrative and in no way limiting.

### EXAMPLE 1

### Materials and Methods

In performing the following syntheses and preparing the following final formulations, the reagents which are used are indicated in the specific examples. Solvents, where used, are preferably distilled prior to use. Sources of other materials are indicated in the appropriate experimental section. In most instances, although not in every instance, trademarked materials are available from Alzo International, Inc., Sayreville, N.J.

Table 1 lists formulations 1-4 that include a cationic polyurethane emulsifier.

The polyurethane emulsifier here comprises the reaction product of 1 mole of behenic acid with 1 mole of triethanolamine that is polymerized by reaction with 1 mole of isophorone diisocyanate and subsequently quaternized by reaction with diethylsulfate.

TABLE 1

| | Formulation | | | |
|---|---|---|---|---|
| | 1 | 2 | 3 | 4 |
| Ingredients | % | % | % | % |
| Mineral Oil (70 ssu) | 10.0 | 10.0 | 10.0 | 10.0 |
| Cationic Polyurethane Emulsifier | 4.0 | 6.0 | 8.0 | 10.0 |
| Deionized Water | 85.0 | 83. | 81.0 | 79.0 |
| Phenobact (antibacterial) | 1.0 | 1.0 | 1.0 | 1.0 |
| | 100.0 | 100.0 | 100.0 | 100.0 |

Formulations 1 and 2 initially formed an emulsion, but were not particularly stable and showed flocculation (creaming) after standing for 2 to 3 hours. Formulation 3 showed slight separation after about eight hours and Formulation 4 showed very slight separation after about 24 hours. The viscosity of these emulsions was determined to be relatively low (<5 cps). Considering that the formulations were made using polymeric emulsifiers, the low viscosity was quite notable. As indicated in Example 2, formulations 5-12, changing the emulsified oil and using a slightly more hydrophilic ester, increased stability.

### EXAMPLE 2

Table 2 lists formulations 5-8 and Table 3 lists formulations 9-12 that include a cationic polyurethane emulsifier as described in Example 1 of the invention.

TABLE 2

| | Formulations | | | |
|---|---|---|---|---|
| | 5 | 6 | 7 | 8 |
| Ingredients | % | % | % | % |
| Isopropyl Palmitate | 10.0 | 10.0 | 10.0 | 10.0 |
| Cationic Polyurethane Emulsifier | 4.0 | 6.0 | 8.0 | 10.0 |
| Deionized Water | 82.0 | 80.0 | 78.0 | 76.0 |
| Glycerin | 3.0 | 3.0 | 3.0 | 3.0 |
| Phenobact | 1.0 | 1.0 | 1.0 | 1.0 |
| | 100.0 | 100.0 | 100.0 | 100.0 |

Formulations 5 and 6 were fairly stable, but showed flocculation after standing for about 4 hours. Formulation 7 showed some flocculation after 2 to 3 weeks, while Formulation 8 was stable, showing no separation after one month's storage at room temperature. The viscosity of these emulsions is surprisingly low (less than 5 cps). Obtaining stability with a low viscosity emulsion is generally very difficult and it is usually achieved using highly charged, water soluble, monomeric, ionic surfactants. The illustrated formulations are believed to be the first stable, low viscosity emulsions made with a polymeric emulsifier. Examples of emulsions made using other oily materials also show very low viscosities, however, the stability is not acceptable. The following emulsions listed in Table 3 all showed flocculation within hours of manufacture.

TABLE 3

| | Formulations | | | |
|---|---|---|---|---|
| | 9 | 10 | 11 | 12 |
| Ingredients | % | % | % | % |
| Soybean oil | 10.0 | 10.0 | — | — |
| Dimethicone (200 cps) | — | — | 10.0 | 10.0 |
| Cationic Polyurethane Emulsifier | 8.0 | 10.0 | 8.0 | 10.0 |
| Deionized Water | 78.0 | 76.0 | 78.0 | 76.0 |
| Glycerin | 3.0 | 3.0 | 3.0 | 3.0 |
| Phenobact | 1.0 | 1.0 | 1.0 | 1.0 |
| | 100.0 | 100.0 | 100.0 | 100.0 |

### EXAMPLE 3

Table 4 lists formulations 13-16 and Table 5 lists formulations 17-18 that include a cationic polyurethane emulsifier as described in Example 1 of the invention.

TABLE 4

| | Formulation | | | |
|---|---|---|---|---|
| | 13 | 14 | 15 | 16 |
| Ingredients | % | % | % | % |
| Isopropyl Palmitate | 10.0 | 10.0 | 10.0 | 10.0 |
| Cationic Polyurethane Emulsifier | 8.0 | 8.0 | 8.0 | 8.0 |
| Deionized Water | 77.6 | 77.5 | 76.5 | 76.5 |
| Glycerin | 3.0 | 3.0 | 3.0 | 3.0 |
| Carbopol 940 | 0.3 | — | — | — |
| Natrosol 250 HR | — | 0.5 | — | — |
| PEG 6000 Distearate | — | — | 1.5 | — |
| Dermothix 100 | — | — | — | 1.5 |
| Triethanolamine | 0.1 | — | — | — |
| Phenobact | 1.0 | 1.0 | 1.0 | 1.0 |
| | 100.0 | 100.0 | 100.0 | 100.0 |

Formulations 13 through 16 use typical polymeric viscosity builders. Carbopol is anionic and complexes with the cationic polyurethane emulsifier to form noticable particles, but the viscosity is not increased. In Formulation 14, Natrosol is nonionic and the viscosity was 600 cps. PEG 6000 Distearate in Formulation 15 is a nonionic viscosity builder (stearic acid diester of 150 mole ethoxylated glycol) and the formulation had a viscosity of 3,300 cps. Dermothix 100 is a urethane dimer of 100 mole ethoxylated stearyl alcohol and the formulation had a 17,500 cps viscosity. Formulations 14, 15 and 16 proved stable after one month's storage at room temperature; without the viscosity builder, floculation occurred in 2-3 weeks (Formulation 7).

Formulations 17 and 18 of Table 5 used conventional viscosity building ingredients. The viscosity of the polymeric emulsions of the present invention can also be increased through the addition of low HLB emulsifiers as described previously. Thus, the following “Emulsifying Wax” examples were prepared using a cationic polyurethane emulsifier as described in Example 1 of the invention.

TABLE 5

Formulation

| | 17 | 18 |
|---|---|---|
| Ingredients | % | % |
| Cationic Polyurethane emulsifier | 16.67 | 25.0 |
| Cetyl Alcohol | 33.33 | 37.5 |
| Stearyl Alcohol | 33.33 | 37.5 |
| Ceteareth 14 | 16.67 | — |
| | 100.00 | 100.0 |

Formulations 17 and 18 are emulsifying waxes that use the polyurethane emulsifier as an o/w emulsifier. Formulation 17 also contains Ceteareth 14 as an auxilliary high HLB nonionic emulsifier. The auxilliary emulsifier can be helpful in forming an emulsion, but it is not a necessary factor. In addition to increasing the viscosity and stability of an emulsion, the use of an emulsifying wax, such as shown in Formulations 17 and 18, reduces the amount of polymeric emulsifier needed to produce a stable emulsion and reduces the overall cost, since the cost of the fatty alcohols is significantly less than that of the polymeric emulsifier.

### EXAMPLE 4

Table 6 lists formulations 19-22 and Table 7 lists formulation 23 that include a cationic polyurethane emulsifier as described in Example 1 of the invention.

TABLE 6

| | Formulations | | | |
|---|---|---|---|---|
| | 19 | 20 | 21 | 22 |
| Ingredients | % | % | % | % |
| Mineral Oil (70 ssu) | 10.0 | 10.0 | 10.0 | 10.0 |
| Emulsifing Wax (Formulation 17) | 2.0 | 4.0 | 6.0 | 8.0 |
| Deionized Water | 87.0 | 85.0 | 83.0 | 81.0 |
| Phenobact | 1.0 | 1.0 | 1.0 | 1.0 |
| | 100.0 | 100.0 | 100.0 | 100.0 |

Formulation 19 separated within one week, probably due to insufficient emulsifier. Formulation 20 was stable at one week and had a viscosity of 4,200 cps. Formulation 21 was stable at one week and had a viscosity of 11,200 cps. Formulation 22 was stable at one week and had a viscosity of 22,000 cps. Formulation 20, 21 and 22 were stable after 6 months storage at room temperature

TABLE 7

| Formulation | 23 |
|---|---|
| Ingredients | % |
| Mineral Oil (70 ssu) | 10.00 |
| Emulsifing Wax (Formulation 18) | 6.00 |
| Deionized Water | 80.88 |
| Glycerin | 2.00 |
| Fragrance | 0.10 |
| FD&C Red #40 (1% aq. solution) | 0.02 |
| Phenobact | 1.00 |
| | 100.00 |

Formulation 23 contained 1.5% of a polymeric emulsifier as described in Example 1 of the invention and 4.5% fatty alcohol combined and used as an emulsifying wax. The viscosity is 23,000 cps and the emulsion was stable and showed no signs of separation after 6 months of room temperature storage. Panel testing of this product proved it to be an acceptable hand lotion, although somewhat oily feeling.
The following formulations listed in Example 5, Table 8, were made to reduce this oily feel.

### EXAMPLE 5

Table 8 lists formulations 24-26 and Table 9 lists formulation 27 that include a cationic polyurethane emulsifier as described in Example 1 of the invention.

TABLE 8

| | Formulation | | |
|---|---|---|---|
| | 24 | 25 | 26 |
| Ingredients | % | % | % |
| Cyclomethicone D5 | 10.0 | — | — |
| Dimethicone (100 cps) | 1.0 | 1.0 | — |
| Isohexyl Decanoate | — | 10.0 | 5.00 |
| Mineral Oil (70 ssu) | — | — | 5.00 |
| Isododecane | — | — | 5.00 |
| Emulsifing Wax (Example 18) | 6.0 | 6.0 | 6.00 |
| Deionized Water | 79.9 | 79.9 | 75.95 |
| Glycerin | 2.0 | 2.0 | 2.00 |
| Color | QS | QS | QS |
| Fragrance | 0.1 | 0.1 | 0.05 |
| Phenobact | 1.0 | 1.0 | 1.00 |
| | 100.0 | 100.0 | 100.00 |

Formulations 24-26 were all less oily feeling than Formulation 23 and were stable. Formulation 24 has a viscosity of −32,000 cps and was stable for 6 months at room temperature. Formulation 25 has a viscosity of −14,000 cps and was stable for 6 months at room temperature. Formulation 26 has a viscosity of −23,000 cps and was stable for 3 months at room temperature.

Formulation 27, Table 9, illustrates the use of a low HLB emulsifier other than a fatty alcohol in combination with the cationic polyurethane emulsifier as described in Example 1 to make an emusifying wax.

TABLE 9

| Formulation | 27 |
|---|---|
| Ingredients | % |
| Mineral Oil (70 ssu) | 10.0 |
| Cationic Polyurethane emulsifier | 1.5 |
| Glyceryl Stearate | 4.5 |
| Deionized Water | 80.9 |
| Glycerin | 2.0 |
| Fragrance | 0.1 |
| FD&C Red #40 (1% aq. solution) | QS |
| Phenobact | 1.0 |
| | 100.0 |

### EXAMPLE 6

Table 10 lists formulation 28 that includes a cationic polyurethane emulsifier as described in Example 1 of the invention. Formulation 28 uses ultraviolet light absorbing esters in combination with the emulsifying wax of the invention to make a sunscreen lotion.

TABLE 10

| Formulation | 28 |
|---|---|
| Ingredients | % |
| Octyl methoxycinnamate | 7.5 |
| Menthyl Anthranilate | 5.0 |
| Isohexyl Decanoate | 7.5 |
| Emulsifing Wax (Formulation 18) | 8.0 |
| Deionized Water | 71.0 |
| Phenobact | 1.0 |
| | 100.0 |

Formulation 28 is a light cream product with a viscosity of 25,000 cps. It has a break point that not readily apparent, but that is typical of a polymeric surfactant. It leaves a somewhat oily film on skin that resists water wash off. Other sunscreen agents such as the benzophenones, avobenzone, zinc oxide or titanium dioxide may also be added to the formulation.

The principles, preferred embodiments and modes of operation of the invention have been described in the foregoing specification. The invention, which is intended to be protected herein, however, is not to be construed as limited to the particular form disclosed, since these are to be regarded as illustrative rather than restrictive. Variations and changes may be made by those skilled in the art without departing from the spirit of the invention.

1. A cationic polyurethane emulsifier formed by:
(a) reacting approximately equimolar amounts of triethanolamine with a fatty acid to form a dihydroxyethyl aminoethyloxy fatty acid ester;
(b) reacting approximately equimolar amounts of the dihydroxyethyl aminoethyloxy fatty acid ester with a diisocyanate to form a urethane polymer; and
(c) reacting the urethane polymer with a quaternizing agent.
wherein the reactions can be performed in one pot or in steps.

2. The cationic polyurethane emulsifier of claim 1, wherein the fatty acid is selected from the group consisting of behenic, erucic, arachidic, gadoleic, stearic, oleic, linoleic, linolenic, hydroxystearic, palmitic, and palmitoleic acids, the diisocyanate is isophorone diisocyanate, and the quaternizing agent is selected from the group consisting of alkyl halides, dimethyl sulfate, and diethyl sulfate.

3. An o/w emulsion, wherein the o/w emulsion comprises:
(1) a polyurethane emulsifier formed by:
(a) reacting approximately equimolar amounts of triethanolamine with a fatty acid to form a dihydroxyethyl aminoethyloxy fatty acid ester;
(b) reacting approximately equimolar amounts of the dihydroxyethyl aminoethyloxy fatty acid ester with a diisocyanate to form a urethane polymer; and
(c) reacting the urethane polymer with quaternizing agent;
(2) a nonaqueous phase comprising one or more of an emollient oil material, and optionally a nonionic or cationic surfactant, or non-polyurethane emulsifier; and
(3) a continuous water phase,
wherein the polyurethane emulsifier disperses the nonaqueous phase in the continuous water phase.

4. The emulsion of claim 3, wherein the fatty acid is selected from the group consisting of behenic, erucic, arachidic, gadoleic, stearic, oleic, linoleic, linolenic, hydroxystearic, palmitic, and palmitoleic acids, and the quaternizing agent is selected from the group consisting of alkyl halides, dimethyl sulfate, and diethyl sulfate.

5. A personal care product comprising a cationic polyurethane emulsifier of claim 1.

6. A personal care product comprising an emulsion of claim 3.

7. A personal care product of claim 5, wherein:
(a) the cationic polyurethane emulsifier is used as a co-emulsifier in an emulsifying wax mixture comprising from about 2% to about 20% by weight of the personal care product; and
(b) the emulsifying wax is comprised of from about 25% to about 90% by weight of low HLB emulsifiers chosen from the group of surfactants having an HLB of less than about 6.

8. A personal care product of claim 7, wherein the low HLB emulsifiers are selected from the group consisting of fatty alcohols, low mole ethoxylates of fatty alcohols, fatty acids, low mole ethoxylates of fatty acids, mono and di glyceryl esters, mono glycol esters, ethoxylated mono and di glyceryl esters, ethoxylated mono glycol esters and fatty amides.

9. A personal care product of claim 5, wherein the cationic polyurethane emulsifier is a co-emulsifier of an emulsion of oily material in water, and wherein the personal care product comprises from about 0.5% to about 5% of the cationic polyurethane emulsifier and from about 1% to about 10% of a w/o emulsifier selected from the group of surfactants having an HLB of less than about 6.

10. A personal care product of claim 9, wherein the w/o emulsifier is selected from the group consisting of fatty alcohols, low mole ethoxylates of fatty alcohols, fatty acids, low mole ethoxylates of fatty acids, mono and di glyceryl esters, mono glycol esters, ethoxylated mono and di glyceryl esters, ethoxylated mono glycol esters, and fatty amides.

11. A personal care product of claim 5, wherein the product is selected from the group consisting of hair conditioners, sunscreens, shampoos, lotions and creams.

12. A personal care product of claim 5, wherein the product contains a thickener.

13. A personal care product of claim 5, wherein the product contains a viscosity controller.

14. A process comprising:
(a) reacting approximately equimolar amounts of triethanolamine with a fatty acid to form a dihydroxyethyl aminoethyloxy fatty acid ester;
(b) reacting approximately equimolar amounts of the dihydroxyethyl aminoethyloxy fatty acid ester with a diisocyanate to form a urethane polymer; and
(c) reacting the urethane polymer with quaternizing agent,
wherein the reaction can occur in a single step in one pot or in more than a single step.

15. A process comprising forming an o/w emulsion comprising:
(1) synthesizing a polyurethane emulsifier by:
(a) reacting approximately equimolar amounts of triethanolamine with a fatty acid to form a dihydroxyethyl aminoethyloxy fatty acid ester;
(b) reacting approximately equimolar amounts of the dihydroxyethyl aminoethyloxy fatty acid ester with a diisocyanate to form a urethane polymer; and
(c) reacting the urethane polymer with quaternizing agent; and
(2) adding the polyurethane emulsifier to a reaction mixture comprising (a) a nonaqueous phase comprising one or more of the following: an emollient oil, nonionic or cationic surfactant, or non-polyurethane emulsifier, and (b) a continuous water phase, thereby dispersing the nonaqueous phase within the continuous water phase.

16. A w/o emulsion, wherein the w/o emulsion comprises:
(1) a polyurethane emulsifier formed by:
(a) reacting approximately equimolar amounts of triethanolamine with a fatty acid to form a dihydroxyethyl aminoethyloxy fatty acid ester;
(b) reacting approximately equimolar amounts of the dihydroxyethyl aminoethyloxy fatty acid ester with a diisocyanate to form a urethane polymer; and
(c) reacting the urethane polymer with quaternizing agent;
(2) a continuous nonaqueous phase comprising one or more of an emollient oil, anionic surfactant, or non-polyurethane emulsifier; and
(3) a water phase,
wherein the polyurethane emulsifier disperses the water phase in the continuous nonaqueous phase.

17. The emulsion of claim 16, wherein the fatty acid is selected from the group consisting of behenic, erucic, arachidic, gadoleic, stearic, oleic, hydroxystearic, palmitic, and palmitoleic acids, and the quaternizing agent is selected from the group consisting of alkyl halides, dimethyl sulfate, and diethyl sulfate. 1

18. A personal care product comprising an emulsion of claim 16.

19. A personal care product of claim 18, wherein:
(a) the cationic polyurethane emulsifier is used as a co-emulsifier in an emulsifying wax mixture comprising from about 10% to about 75% by weight of the personal care product; and
(b) the personal care product is comprised of from about 25% to about 90% by weight of low HLB emulsifiers chosen from the group of surfactants having an HLB of less than about 6.

20. A personal care product of claim 18, wherein the low HLB emulsifiers are selected from the group consisting of fatty alcohols, low mole ethoxylates of fatty alcohols, fatty acids, low mole ethoxylates of fatty acids, mono and di glyceryl esters, mono glycol esters, ethoxylated mono and di glyceryl esters, ethoxylated mono glycol esters and fatty amides.

21. A personal care product of claim 18, wherein the cationic polyurethane emulsifier is a co-emulsifier of an emulsion of oily material in water, and wherein the personal care product comprises from about 0.5% to about 5% of the cationic polyurethane emulsifier and from about 1% to about 10% of an emulsifier selected from the group of surfactants having an HLB of less than about 6.

22. A personal care product of claim 18, wherein the emulsifier is selected from the group consisting of fatty alcohols, low mole ethoxylates of fatty alcohols, fatty acids, low mole ethoxylates of fatty acids, mono and di glyceryl esters, mono glycol esters, ethoxylated mono and di glyceryl esters, ethoxylated mono glycol esters, and fatty amides.

23. A personal care product of claim 18, wherein the product is selected from the group consisting of hair conditioners, sunscreens, or shampoos.

24. A personal care product of claim 18, wherein the product contains a thickener.

25. A personal care product of claim 5, wherein the product contains a viscosity controller.

26. A process comprising forming a w/o emulsion comprising:
(1) synthesizing a polyurethane emulsifier by:
(a) reacting approximately equimolar amounts of triethanolamine with a fatty acid to form a dihydroxyethyl aminoethyloxy fatty acid ester;
(b) reacting approximately equimolar amounts of the dihydroxyethyl aminoethyloxy fatty acid ester with a diisocyanate to form a urethane polymer; and
(c) reacting the urethane polymer with quaternizing agent; and
(2) adding the polyurethane emulsifier to a reaction mixture comprising (a) a continuous nonaqueous phase comprising one or more of the following: an emollient oil, anionic surfactant, or non-polyurethane emulsifier, and (b) a water phase, thereby dispersing the water phase in the continuous nonaqueous phase.
